(19) United States
(12) Patent Application Publication
(10) Pub. No.: US 2025/0251382 A1
(43) Pub. Date: Aug. 7, 2025

(54) INSTRUMENTATION FOR PROCESSING CARBONATE SAMPLES FOR ISOTOPE ANALYSIS

(71) Applicant: Saudi Arabian Oil Company, Dhahran (SA)

(72) Inventors: Feng Hu Lu, Dhahran (SA); Hassan Buri, Dhahran (SA); Rakan Khaldi, Dhahran (SA); Abdullah Hajri, Dhahran (SA); Salman Mohammed Qathami, Dhahran (SA)

(21) Appl. No.: 18/432,655

(22) Filed: Feb. 5, 2024

(51) Int. Cl. G01N 33/24; G01N 1/28; G01N 1/44; H01J 49/04

(52) U.S. Cl. CPC G01N 33/24; G01N 1/286; G01N 1/44; H01J 49/0413

(57) ABSTRACT

A system and method for measuring isotopes in gas released from a rock sample. An exemplary instrument includes a sample tray, which includes an opening to hold a sample vial, wherein the sample vial projects into an fluidically enclosed space in the sample tray, and a heat exchange fluid in the fluidically enclosed space, wherein the heat exchange fluid is in fluidic contact with an outside surface of the sample vial. The sample tray includes a heater to heat the heat exchange fluid in the fluidically enclosed space. The instrument includes an autosampler, including a loop injection interface, including a needle to pierce a septum on the sample vial, a fluidic coupling to a purge gas line, and a fluidic coupling to a mass spectrometer. The mass spectrometer to measure the isotopes in a gas released from the rock sample.

### TECHNICAL FIELD

This disclosure relates to automated methods for analyzing carbonate samples for isotope analysis.

### BACKGROUND

Carbonate samples are analyzed for carbon and oxygen isotopes utilizing a mass spectrometer coupled to an autosampler taking gas samples from glass vials in a heated tray. The tray is configured to facilitate a carbonate-acid reaction in the glass vials by heating the glass vials, for example, to about 75° C., and to maintain a steady temperature with minor variation, for example, less than 1° C./hour, during analysis. In reality, different temperatures are observed between spots within the heated tray, for example, a difference of 1.5° C., due to different heating capacities of the heating bars at the bottom of the tray. Moreover, the heating bars may not transfer heat homogenously to all spots.

### SUMMARY

An embodiment described herein provides an instrument for measuring isotopes in gas released from a rock sample. The instrument includes a sample tray, which includes an opening to hold a sample vial, wherein the sample vial projects into an fluidically enclosed space in the sample tray, a heat exchange fluid in the fluidically enclosed space, wherein the exchange fluid is in fluidic contact with an outside surface of the sample vial. The sample tray includes a heater to heat the heat exchange fluid in the fluidically enclosed space. The instrument includes an autosampler, including a loop injection interface, including a needle to pierce a septum on the sample vial, a fluidic coupling to a purge gas line, and a fluidic coupling to a mass spectrometer. The mass spectrometer to measure the isotopes in a gas released from the rock sample.

Another embodiment described in examples herein provides a method for measuring isotopes in gas released from a rock sample. The method includes placing a rock sample in a sample vial, sealing the sample vial with a septum, placing the sample vial in a sample tray. The sample tray includes an opening to hold the sample vial, wherein the sample vial projects into an fluidically enclosed space in the sample tray, a heat exchange fluid in the fluidically enclosed space, wherein the exchange fluid is in fluidic contact with an outside surface of the sample vial, and a heater to heat the heat exchange fluid in the fluidically enclosed space. The method also includes piercing the septum with a needle from an autosampler, purging the sample vial with a purge gas, and injecting an acid reactant into the sample vial. The heat exchange fluid surrounding the outside surface of the sample vial is heated to between about 70° C. and about 75° C. Gas formed in the sample vial is swept into a mass spectrometer. Amounts of carbon isotopes and oxygen isotopes released from the rock sample are measured.

### BRIEF DESCRIPTION OF DRAWINGS

FIG. 1A is a schematic drawing of an autosampler tray heater 100 that uses a liquid to evenly heat sample vials.

FIG. 1B is a drawing of a lid 112 that covers the autosampler tray heater.

FIG. 2 is an autosampler vial 200 that reduces dead volume for improved purging.

FIG. 3 is a method 300 for processing carbonate samples for isotope analysis using the autosampler tray heater and autosampler vial.

### DETAILED DESCRIPTION

Carbon and oxygen isotopes of carbonates can be measured to determine paleoenvironmental conditions, and correlate reservoirs with source rocks. For example, an autosampler and thermal tray with a sample preparation and loop injection interface, such as the Thermo Scientific GasBench II with the Carbonate-Option, can be coupled to a mass spectrometer, such as the Thermo Scientific isotope ratio mass spectrometer, for carbon and oxygen isotope analysis. The thermal tray holds glass vials with powder samples. The thermal tray has a heating unit, which is a heating element placed at the bottom of the tray, to raise the temperature of the glass vials up to between about 70° C. and 75° C. to accelerate a reaction between phosphoric acid and carbonate powder in the individual sample vials.

Before the acid is added to a vial containing a sample powder, the vial holding the sample powder is purged, for example, with helium gas, to remove air which includes CO₂. It takes about 10 minutes to completely purge a vial, as helium is sequentially introduced and removed to replace the air out of the vial. Once the acid is introduced into the vial, the reaction takes about two hours while the tray is at about 70° C., or 48 hours if the tray stays at room temperature. The evolved CO₂ from the reaction is then swept in a stream of the purge gas, e.g., helium, to the mass spectrometer for isotope analysis.

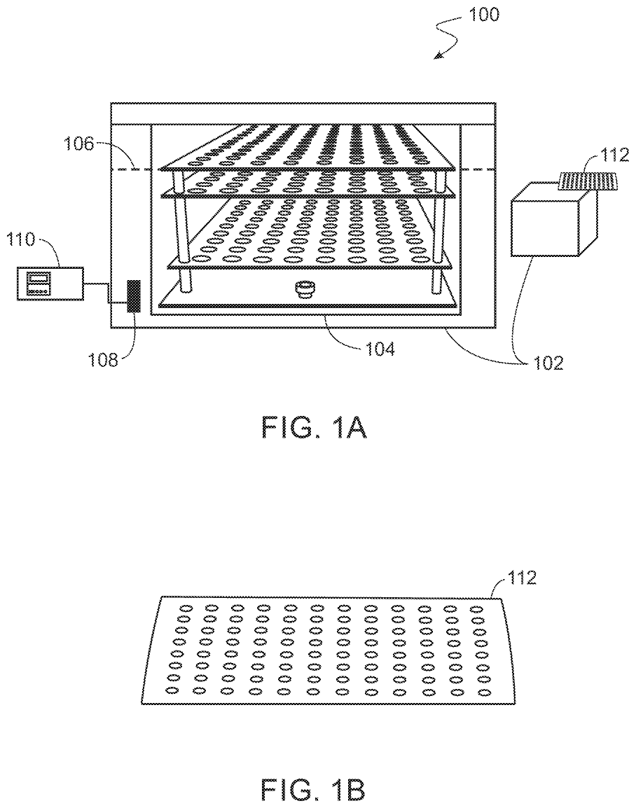

FIG. 1A is a schematic drawing of an autosampler tray heater 100 that uses a liquid to evenly heat sample vials. The autosampler tray heater 100 includes a water box 102 that holds a sample vial rack 104. Water is added to the water box 102 until the water level 106 reaches the top of the sample vial rack 104. A heater 108 is placed in the water box 102 to raise the temperature of the water. In some embodiments, the heater 108 includes heating elements and a water circulator.

In some embodiments, the water box 102 matches the size of a current heating tray, for example, about 30 cm wide, about 80 cm long, and about 60 cm high. The materials for the box, lid, supporter/holder for vials may be plastics and/or metals.

For accurate analysis, the temperature of the sample vials should be within 0.25° C., as the temperature is particularly important for isotope fractionation. Further, a steady temperature, with any changes at less than 1° C. per hour. Further, it is assumed for the calculations that all samples including standards are analyzed at the same conditions, including temperature. If the temperature is not the same for all samples, the isotope shift may not be from the sample, but derived from the change of temperature with the sample tray. Therefore, the quality of analysis is compromised.

Accordingly, in some embodiments, the heater 108 is coupled to a heating control unit 110, such as a Jumo meter, that measures the temperature of the water and controls it to the desired limits, for example, to within about 0.1° C. of the setpoint, within about 0.25° C. of a set point, within about 0.5° C. of the setpoint, or within about 1.0° C. of the setpoint. Depending on the sample and the acid, the setpoint may be between about 60° C. and about 80° C., about 70° C. and about 75° C., within about 72° C. and about 74° C., about 70° C., about 71° C., about 72° C., about 73° C., about 74° C., about 75° C., or higher.

FIG. 1B is a drawing of a lid 112 that covers the autosampler tray heater 100. The lid 112 has holes for sample vials that correspond to the holes in the sample vial rack 104. The lid 112 decreases water evaporation from the water box 102. In some embodiments, the liquid in the water box 102 is not water, but mineral oil. This allows a higher temperature to be used for analyzing samples with different chemistry.

The sample vials extend from the lid 112 into the water box 102. Accordingly, shorter sample vials may not be heated correctly, even though there is a significant amount of dead volume that must be purged in the current sample vials.

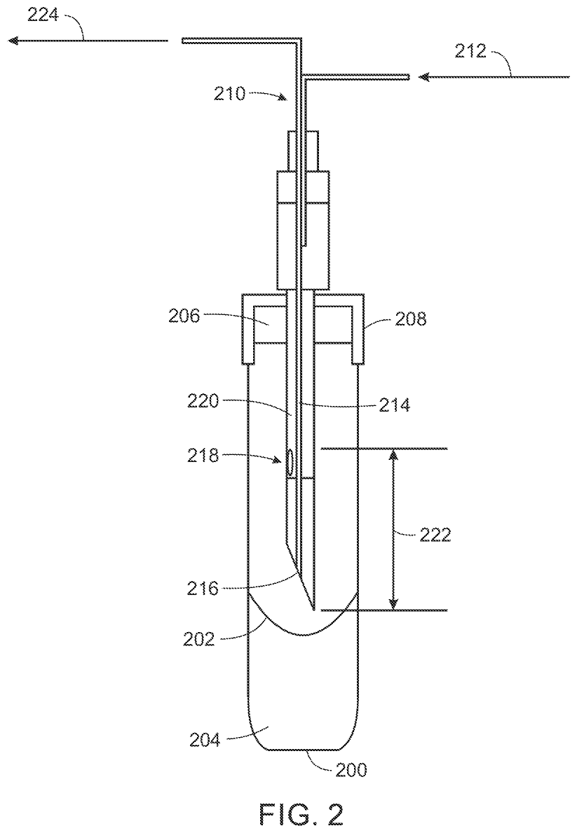

FIG. 2 is an autosampler vial 200 that reduces dead volume for improved purging. In this embodiment, a base 202 is included in the autosampler vial 200 to cut off the dead volume space 204 at the base of the autosampler vial 200. A septum 206, for example, made of Teflon, polyurethane, or other materials, is sealed to the top of the autosampler vial 200, for example, with a cap 208 that is attached to the autosampler vial 200 with threads or is crimped to the autosampler vial 200. A transfer needle 210 is used by the autosampler to pierce the septum 206 for processing a sample. During purging and sampling, a helium stream 212 is introduced into the transfer needle 210, flowing through a center conduit 214 and exiting the transfer needle 210 through a hole 216 near the tip of the transfer needle 210. The helium flows through the autosampler vial 200 and exits through an opening 218 into the side of the transfer needle 210, which allows the helium to flow through an annulus 220 around the center conduit 214. The opening 218 is separated from the tip of the needle by about 12 mm, as shown by reference number 222. The outlet stream 224 then exits the transfer needle 210, for example, to waste, if during purging, or to the mass spectrometer, if during sampling.

Without the base 202, the transfer needle 210 would only penetrate about one half the depth of the autosampler vial 200, and the entire autosampler vial including the dead volume space 204 would need to be purged. Including the base 202 increases the efficiency of the purging as the autosampler vial 200 is around 10 cm in length, and the base 202 would be incorporated about 5 cm to about 6 cm down from the top, decreasing the volume needing to be purged by about 50%. In some embodiments, the base 202 is a glass surface that is formed with the autosampler vial 200 during manufacturing. In other embodiments, the base 202 is a plastic or rubber plug that is pushed into the autosampler vial 200. Further, the detection limit will be increased since the decrease in volume or proportionally increased the relative concentration of the CO₂.

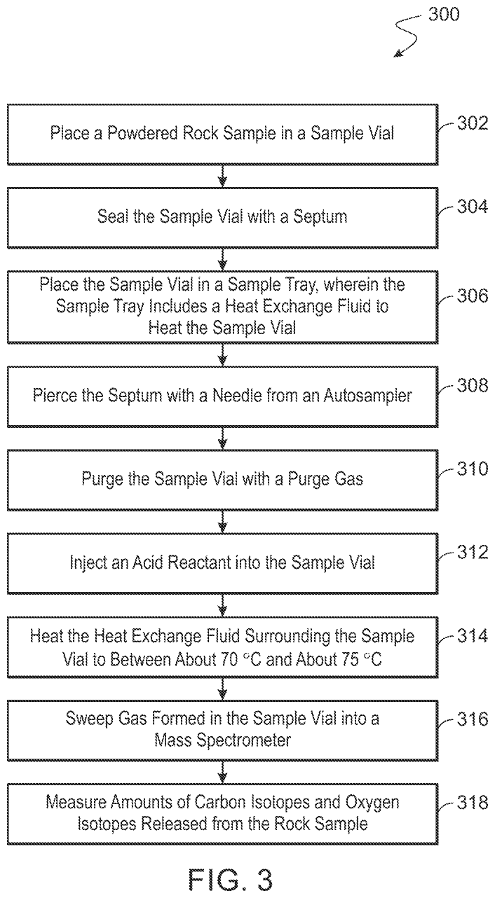

FIG. 3 is a method 300 for processing carbonate samples for isotope analysis using the autosampler tray heater and autosampler vial. The method begins at block 302, when a powder rock sample is placed in a sample vial. At block 304, the sample vial is sealed with the septum, for example, mounted in a cap that is crimped onto the sample vial.

At block 306 the sample vial is placed in the sample tray, wherein the sample tray includes a heat exchange fluid to heat the sample vial. As described above, the heat exchange fluid may be water or mineral oil, depending on the temperature desired for the sample vial.

At block 308, the septum is pierced with a needle. At block 310, the sample vial is purged with a purge gas. As described herein, the purge gas can be helium. While the purging is generally performed by the autosampler, the purging can be performed manually.

After purging, at block 312, an acid reactant is injected into the sample vial. For the carbon isotope and oxygen isotope analysis, the acid reactant is phosphoric acid. In some embodiments, the acid reactant is injected automatically using the autosampler. For example, the autosampler can have two needles that are separated with space so that one needle that is coupled to the purge gas, as described with respect to FIG. 1A, purges a first row of samples, while the second needle that is coupled to an acid reservoir adds acid to a previously purged row of samples in the sample tray. In other embodiments, the acid is added manually. In these embodiments, the autosampler is allowed to purge the vials, then a manual syringe is used to inject acid into each of the vials.

At block 314, the heat exchange fluid surrounding the sample vial is heated to accelerate the reaction. For example, the heat exchange fluid is heated to between about 70° C. and about 75° C. At block 316, the gas formed in the sample vial is swept from the sample vial by the purge gas into a mass spectrometer.

At block 318, the mass spectrometer measures the amounts carbon isotopes and oxygen isotopes in the gas stream from the sample vial. The ratios of the isotopes can then be used to determine paleoenvironmental conditions, and correlate reservoirs with source rocks. For example, measurements of the isotopes can be used to determine the ratio of ¹³C/¹²C and ratio of ¹⁸O/¹⁶O in the CO₂ released from the reaction between carbonate powder and acid in the vial. In geological history, carbon cycling (land erosion versus sea deposition and burial) results in fractionation of ¹³C and ¹²C. The isotope ¹²C prefers to stay with organic matter. Temperature fluctuations of the surface of the earth causes fractionation of ¹⁸O and ¹⁶O, for example, cycles between global glaciation and global warming will shift the ratio of ¹⁸O and ¹⁶O in seawater forward and backward. The shifts/variations of carbon and oxygen isotope ratios will be recorded while minerals are deposited in different geological times. Thus, the measurements of C and O isotopes in carbonate rocks can be utilized to re-construct ancient environment and climate.

### EMBODIMENTS

An embodiment described herein provides an instrument for measuring isotopes in gas released from a rock sample. The instrument includes a sample tray, which includes an opening to hold a sample vial, wherein the sample vial projects into an fluidically enclosed space in the sample tray, a heat exchange fluid in the fluidically enclosed space, wherein the exchange fluid is in fluidic contact with an outside surface of the sample vial. The sample tray includes a heater to heat the heat exchange fluid in the fluidically enclosed space. The instrument includes an autosampler, including a loop injection interface, including a needle to pierce a septum on the sample vial, a fluidic coupling to a purge gas line, and a fluidic coupling to a mass spectrometer. The mass spectrometer to measure the isotopes in a gas released from the rock sample.

In an aspect, combinable with any other aspect, the heater includes a heating element, a temperature controller, and a fluid circulator.

In an aspect, combinable with any other aspect, the sample tray includes a fluid inlet into the fluidically enclosed space, wherein the fluid inlet is coupled to an outlet on a circulating fluid heater, and a fluid outlet from the fluidically enclosed space, wherein the fluid outlet is coupled to an inlet on the circulating fluid heater. In an aspect, the circulating fluid heater includes a reservoir for the heat exchange fluid, a heater to heat the heat exchange fluid, a temperature controller, and a pump to circulate the heat exchange fluid between the outlet and the inlet.

In an aspect, the heat exchange fluid includes water. In an aspect, the heat exchange fluid includes mineral oil.

In an aspect, combinable with any other aspect, the heat exchange fluid is at a temperature of about 75° C.

In an aspect, combinable with any other aspect, the purge gas line is fluidically coupled to a solenoid valve, and wherein the solenoid valve is fluidically coupled to a gas cylinder. In an aspect, a gas in the gas cylinder includes helium.

In an aspect, combinable with any other aspect, the instrument includes a sample vial, wherein the sample vial includes a cylindrical glass body. The cylindrical glass body includes a longitudinal axis perpendicular to a diameter of the cylindrical glass body, wherein the longitudinal axis is greater than the diameter, an opening at one end of the longitudinal axis, and a seal at the opposite end of the longitudinal axis from the opening, wherein the seal extends up the longitudinal axis from the opposite end to about one half of a length to the opening. In an aspect, the instrument includes a rock sample, and a septum sealing the opening. In an aspect, the instrument includes a reactant in the sample vial, wherein the reactant includes an acid. In an aspect, the sample vial includes a gas including carbon isotopes and oxygen isotopes released from the rock sample.

Another embodiment described in examples herein provides a method for measuring isotopes in gas released from a rock sample. The method includes placing a rock sample in a sample vial, sealing the sample vial with a septum, placing the sample vial in a sample tray. The sample tray includes an opening to hold the sample vial, wherein the sample vial projects into an fluidically enclosed space in the sample tray, a heat exchange fluid in the fluidically enclosed space, wherein the exchange fluid is in fluidic contact with an outside surface of the sample vial, and a heater to heat the heat exchange fluid in the fluidically enclosed space. The method also includes piercing the septum with a needle from an autosampler, purging the sample vial with a purge gas, and injecting an acid reactant into the sample vial. The heat exchange fluid surrounding the outside surface of the sample vial is heated to between about 70° C. and about 75° C. Gas formed in the sample vial is swept into a mass spectrometer. Amounts of carbon isotopes and oxygen isotopes released from the rock sample are measured.

In an aspect, combinable with any other aspect, the method includes determining paleoenvironmental conditions from the amounts of carbon isotopes and oxygen isotopes released from the rock sample.

In an aspect, combinable with any other aspect, the method includes correlating reservoir rocks and source rocks based, at least in part, on the amounts of carbon isotopes and oxygen isotopes released from the rock sample.

In an aspect, combinable with any other aspect, the method includes forming the rock sample by crushing a carbonate rock sample from a reservoir.

In an aspect, combinable with any other aspect, the method includes forming the sample vial, wherein the sample vial includes a cylindrical glass body. The cylindrical glass body includes a longitudinal axis perpendicular to a diameter of the cylindrical glass body, wherein the longitudinal axis is greater than the diameter, an opening at one end of the longitudinal axis, and a seal at the opposite end of the longitudinal axis from the opening, wherein the seal extends up the longitudinal axis from the opposite end to about one half of a length to the opening.

In an aspect, combinable with any other aspect, the method includes heating the heat exchange fluid for about two hours before sweeping the gas formed in the rock sample into the mass spectrometer.

In an aspect, combinable with any other aspect, the method includes maintaining temperature of a plurality of sample vials in the sample tray within about 0.5° C. of each other.

Other implementations are also within the scope of the following claims.

What is claimed is:

1. An instrument for measuring isotopes in gas released from a rock sample, comprising:
a sample tray, comprising:
an opening to hold a sample vial, wherein the sample vial projects into an fluidically enclosed space in the sample tray;
a heat exchange fluid in the fluidically enclosed space, wherein the heat exchange fluid is in fluidic contact with an outside surface of the sample vial; and
a heater to heat the heat exchange fluid in the fluidically enclosed space;
an autosampler, comprising a loop injection interface, comprising:
a needle to pierce a septum on the sample vial;
a fluidic coupling to a purge gas line; and
a fluidic coupling to a mass spectrometer; and
the mass spectrometer to measure the isotopes in a gas released from the rock sample.

2. The instrument of claim 1, wherein the heater comprises:
a heating element;
a temperature controller; and
a fluid circulator.

3. The instrument of claim 1, wherein the sample tray comprises:
a fluid inlet into the fluidically enclosed space, wherein the fluid inlet is coupled to an outlet on a circulating fluid heater; and
a fluid outlet from the fluidically enclosed space, wherein the fluid outlet is coupled to an inlet on the circulating fluid heater.

4. The instrument of claim 3, wherein the circulating fluid heater comprises:
a reservoir for the heat exchange fluid;
a heater to heat the heat exchange fluid;
a temperature controller; and
a pump to circulate the heat exchange fluid between the outlet and the inlet.

5. The instrument of claim 1, wherein the heat exchange fluid comprises water.

6. The instrument of claim 1, wherein the heat exchange fluid comprises mineral oil.

7. The instrument of claim 1, wherein the heat exchange fluid is at a temperature of about 75° C.

8. The instrument of claim 1, wherein the purge gas line is fluidically coupled to a solenoid valve, and wherein the solenoid valve is fluidically coupled to a gas cylinder.

9. The instrument of claim 8, wherein a gas in the gas cylinder comprises helium.

10. The instrument of claim 1, comprising a sample vial, wherein the sample vial comprises a cylindrical glass body, comprising:
a longitudinal axis perpendicular to a diameter of the cylindrical glass body, wherein the longitudinal axis is greater than the diameter;
an opening at one end of the longitudinal axis; and
a seal at the opposite end of the longitudinal axis from the opening, wherein the seal extends up the longitudinal axis from the opposite end to about one half of a length to the opening.

11. The instrument of claim 10, comprising:
a rock sample; and
a septum sealing the opening.

12. The instrument of claim 11, comprising a reactant in the sample vial, wherein the reactant comprises an acid.

13. The instrument of claim 12, wherein the sample vial comprises a gas comprising carbon isotopes and oxygen isotopes released from the rock sample.

14. A method for measuring isotopes in gas released from a rock sample, comprising:
placing a rock sample in a sample vial;
sealing the sample vial with a septum;
placing the sample vial in a sample tray, wherein the sample tray comprises:
an opening to hold the sample vial, wherein the sample vial projects into an fluidically enclosed space in the sample tray;
a heat exchange fluid in the fluidically enclosed space, wherein the heat exchange fluid is in fluidic contact with an outside surface of the sample vial; and
a heater to heat the heat exchange fluid in the fluidically enclosed space;
piercing the septum with a needle from an autosampler;
purging the sample vial with a purge gas;
injecting an acid reactant into the sample vial;
heating the heat exchange fluid surrounding the outside surface of the sample vial to between about 70° C. and about 75° C.;
sweeping gas formed in the sample vial into a mass spectrometer; and
measuring amounts of carbon isotopes and oxygen isotopes released from the rock sample.

15. The method of claim 14, comprising determining paleoenvironmental conditions from the amounts of carbon isotopes and oxygen isotopes released from the rock sample.

16. The method of claim 14, comprising correlating reservoir rocks and source rocks based, at least in part, on the amounts of carbon isotopes and oxygen isotopes released from the rock sample.

17. The method of claim 14, comprising forming the rock sample by crushing a carbonate rock sample from a reservoir.

18. The method of claim 14, comprising forming the sample vial, wherein the sample vial comprises a cylindrical glass body, comprising:
a longitudinal axis perpendicular to a diameter of the cylindrical glass body, wherein the longitudinal axis is greater than the diameter;
an opening at one end of the longitudinal axis; and
a seal at the opposite end of the longitudinal axis from the opening, wherein the seal extends up the longitudinal axis from the opposite end to about one half of a length to the opening.

19. The method of claim 14, comprising heating the heat exchange fluid for about two hours before sweeping the gas formed in the rock sample into the mass spectrometer.

20. The method of claim 14, comprising maintaining temperature of a plurality of sample vials in the sample tray within about 0.5° C. of each other.
